(19) United States
(12) Patent Application Publication
(10) Pub. No.: US 2025/0275931 A1
(43) Pub. Date: Sep. 4, 2025

(54) TOPICAL PATCH COMPOSITION FOR TARGETED SIRNA NANOPLEX DELIVERY AND GENE THERAPIES AND METHOD OF MAKING AND USE THEREOF

(71) Applicants: Nanyang Technological University, Singapore (SG); National Skin Centre (Singapore) Pte Ltd, Singapore (SG)

(72) Inventors: Thatt Yang Timothy TAN, Singapore (SG); Yong Yao CHUN, Singapore (SG); Nguan Soon TAN, Singapore (SG); Hong Liang TEY, Singapore (SG)

(21) Appl. No.: 18/261,891

(22) PCT Filed: Jan. 21, 2022
(86) PCT No.: PCT/SG2022/050027

§ 371 Date: Jul. 18, 2023

(30) Foreign Application Priority Data
Jan. 21, 2021 (SG) 10202100655R

(51) Int. Cl. A61K 9/70; A61K 9/00; A61K 47/36; A61K 47/42; A61K 47/64; A61P 17/02; C12N 15/113

(52) U.S. Cl. CPC A61K 9/7092; A61K 9/0021; A61K 47/36; A61K 47/42; A61K 47/64; A61P 17/02; C12N 15/113; C12N 2310/14

(57) ABSTRACT

Provided herein is a dissolvable microneedle patch comprising a tip portion, wherein the tip portion comprises a nanoplex formed from a positively charged biopolymer which incorporates a cell targeting moiety and a siRNA. In one embodiment, the positively charged biopolymer is formed from gelatin and tyramine, the cell targeting moiety is gelatin, and the siRNA is siSPARC. Also provided herein is the use of the dissolvable microneedle patch in therapy, specifically in the treatment of a skin condition or disorder, such as scarring (e.g. hypertrophic scarring), keloid formation, eczema, or hyperpigmentation.

### INCORPORATION OF ELECTRONIC SEQUENCE LISTING

A sequence listing contained in the file named “MRKSN063USP_Sequence_Listing_ST25.txt,” which is 5,354 bytes and created on Jan. 18, 2024, is filed electronically herewith and incorporated by reference in its entirety.

### FIELD OF INVENTION

The current invention relates to the composition, method of making and use of a topical patch for targeted siRNA nanoplex delivery and gene therapies to treat a skin disorder or condition.

### BACKGROUND

The listing or discussion of a prior-published document in this specification should not necessarily be taken as an acknowledgement that the document is part of the state of the art or is common general knowledge.

Current therapy options for skin disorders such as hypertrophic scar, keloid, eczema, psoriasis or acne is limited due to the challenge of the skin barrier. Further, hydrophilic drugs or therapeutic biomolecules have low skin permeability due to the skin barrier of the stratum corneum, making it difficult for them to reach the epidermis or the dermis layer for treatment (Xie, Y. et al., J. Mater. Chem. B 2019, 7, 6604-6611). Even if the drugs or therapeutic molecules are able to penetrate the stratum corneum, they have to be delivered to the targeted cells at different regions of the skin. Currently, there is no technology or method that could achieve simultaneous drug penetration of skin barrier, delivery to a specific region or depth of the skin where the targeted cells reside, and targeting of the desired cells.

RNA interference (RNAi) is a gene silencing mechanism triggered by small interfering RNAs (siRNA). Short double-stranded RNAs with 21 to 23 nucleotides can provide sequence-specific gene therapy to many undruggable genes and thus can be efficiently designed and targeted for any diseases (Subhan, M. A. & Torchilin, V. P., Nanomed: Nanotechnol. Biol. Med. 2020, 29, 102239). siRNA has been used to treat various skin disorders caused by abnormal gene expression of different cell types found in the different regions of the skin, including alopecia, psoriasis, cancer, inflammation, hyperpigmentation, hypertrophic scars and keloids. Because naked siRNAs are hydrophilic, negatively charged and highly susceptible to degradation by serum and RNases, the main obstacles for their delivery through skin to the targeted cells include 1) the high barrier to deliver through the outermost stratum corneum of the skin, 2) instability through the various layers of the skin such as the epidermis and dermis to reach the desired cells, and 3) inability to target the desired cell due to the lack of a targeting moiety.

Recently, siRNA therapy such as using siRNA to silence connective tissue growth factor (CTGF/CCN-2) or transforming growth factor-β (TGF-β), has gained interest in the prevention and treatment of fibrosis (Kang, S. et al., Nanoscale 2020, 12, 6385-6393; Zhou, J. et al., Oncotarget 2017, 8, 80651-80665; and Cho, K.-H. et al., Tissue Eng. Regen. Med. 2017, 14, 211-220). However, most of the siRNA delivery vehicles are in particle form, which needs a method of administration to penetrate through the skin barrier.

Pathological scars, such as hypertrophic scars and keloids, are chronic skin disorders classified by prolonged and abnormal extracellular matrix accumulation (Payapvipapong, K. et al., J. Cosmet. Dermatol. 2015, 14, 83-90; and Bombaro, K. M. et al., Burns 2003, 29, 299-302). Hypertrophic scars and keloids are resultant of excessive wound healing with prolonged and more vigorous inflammation stages that encourage skin fibrosis (Ellis, S., Lin, E. J. & Tartar, D., Curr. Dermatol. Rep. 2018, 7, 350-358). The development of hypertrophic scars and keloids often cause itchiness, pain, and functional impairment. Further, the disfiguring appearance caused by these chronic skin diseases might lead to emotional distress and psychosocial burden to patients.

Current treatments for hypertrophic scars and keloids are limited and not effective. Non-surgical approaches such as multiple intralesional injections of corticosteroid (Tan, C. W. et al., Dermatol. Ther. 2019, 9, 601-611) or bleomycin (Saitta, P., Krishnamurthy, K. & Brown, L. H., Dermatol. Surg. 2008, 34, 1299-1313) are considered the first-line option and more preferred than painful and expensive surgical procedures. However, long periods and multiple administrations of intralesional injection would cause pain and swelling to the injection site, which will lead to poor patient compliance (Xie, Y. et al., J. Mater. Chem. B 2019, 7, 6604-6611). Also, there is a high recurrence rate when intralesional injection cease (Kelly, A. P., Dermatol. Ther. 2004, 17, 212-218). Other administration routes such as transdermal delivery, are limited by the skin barrier of stratum corneum, especially for hydrophilic drugs such as bleomycin (Xie, Y. et al., J. Mater. Chem. B 2019, 7, 6604-6611).

Thus, there is a need to develop a more effective and pain-free treatment for hypertrophic scars and keloids, which can also overcome the challenges stated above.

### SUMMARY OF INVENTION

The current invention presents a platform technology to overcome the abovementioned obstacles, provide effective siRNA delivery to a desired region in the skin and achieve specific cell-targeting gene silencing, and describes the composition, method of making and use thereof.

Aspects and embodiments of the invention will now be discussed by reference to the following numbered clauses.

1. A dissolvable microneedle patch, comprising:
a substrate portion comprising a first biocompatible material; and
a plurality of microneedle structures extending from the substrate portion, where each microneedle structure comprises:
   a base portion comprising a second biocompatible material; and
   a tip portion, wherein the tip portion comprises:
      a nanoplex formed from a positively charged biopolymer that incorporates a cell targeting moiety and an siRNA; and
      a third biocompatible material.

2. The dissolvable microneedle patch according to Clause 1, wherein the first biocompatible material is selected from one or more of the group consisting of hyaluronic acid, or other dissolvable and biocompatible polymers such as polysaccharides (chitosan, sodium alginate, dextran, and sodium chondroitin sulphate), cellulose derivatives (carboxymethyl cellulose, hydroxypropyl cellulose, and hydroxypropyl methylcellulose) and synthetic polymers (polyvinyl alcohol, polyvinylpyrrolidone, and methylvinylether-co-maleic anhydride).

3. The dissolvable microneedle patch according to Clause 1 or Clause 2, wherein the second biocompatible material is selected from one or more of the group consisting of hyaluronic acid, or other dissolvable and biocompatible polymers such as polysaccharides (chitosan, sodium alginate, dextran, and sodium chondroitin sulphate), cellulose derivatives (carboxymethyl cellulose, hydroxypropyl cellulose, and hydroxypropyl methylcellulose) and synthetic polymers (polyvinyl alcohol, polyvinylpyrrolidone, and methylvinylether-co-maleic anhydride).

4. The dissolvable microneedle patch according to any one of the preceding clauses, wherein the first and the second biocompatible materials are the same biocompatible material.

5. The dissolvable microneedle patch according to any one of the preceding clauses, wherein the third biocompatible material is selected from one or more of the group consisting of hyaluronic acid, or other dissolvable and biocompatible polymers such as polysaccharides (chitosan, sodium alginate, dextran, and sodium chondroitin sulphate), cellulose derivatives (carboxymethyl cellulose, hydroxypropyl cellulose, and hydroxypropyl methylcellulose) and synthetic polymers (polyvinyl alcohol, polyvinylpyrrolidone, and methylvinylether-co-maleic anhydride), optionally wherein the third biocompatible material used to form the tip portion is hyaluronic acid.

6. The dissolvable microneedle patch according to any one of the preceding clauses, wherein the first, second and third biocompatible materials are the same biocompatible material.

7. The dissolvable microneedle patch according to any one of the preceding clauses, wherein the positively charged biopolymer is formed from:
a heterobifunctional polymer comprising a first set of functional groups that are positively charged and a second set of functional groups that are negatively charged in an aqueous environment; and
a molecule conjugated to the heterobifunctional polymer, where the molecule is conjugated by a functional group that forms a bond with the second set of functional groups of the heterobifunctional polymer, so as to reduce the number of the second functional groups in the second set of functional groups, thereby providing the positively charged biopolymer.

8. The dissolvable microneedle patch according to Clause 7, wherein the heterobifunctional polymer is selected from one or more of gelatin, collagen, silk fibroin, elastin and H₂N-PEG-CO₂H.

9. The dissolvable microneedle patch according to Clause 8, wherein the heterobifunctional polymer is gelatin.

10. The dissolvable microneedle patch according to any one of Clauses 7 to 9, wherein the functional group in the molecule conjugated to the heterobifunctional polymer that forms a bond with the second set of functional groups of the heterobifunctional polymer is selected from one or more of amide, amino, and ammonium.

11. The dissolvable microneedle patch according to any one of Clauses 7 to 9, wherein the molecule conjugated to the heterobifunctional polymer is selected from one or more of 4-hydroxybenzylamine, dopamine, and tyramine (e.g. tyramine).

12. The dissolvable microneedle patch according to any one of the preceding clauses, wherein the cell targeting moiety is selected from one or more of gelatin, transferrin, cyclic-Arginine-Glycine-Aspartic acid (RGD) tripeptide, skin protein-derived peptides (e.g. one or more of the group consisting of KTTKS (SEQ ID NO: 11), NAP-amide, and Lam332), and folic acid.

13. The dissolvable microneedle patch according to any one of the preceding clauses, wherein the siRNA is selected from one or more of the group consisting of:

```
(a) siTGFβ1-337:
  (SEQ ID NO: 1)
5′-GGUGGAAACCCACAACGAATT-3′;
(b) SIFAK:
(SEQ ID NO: 2)
5′-GCGAAATCCA-TAGCAGGCCACTATAGTGAGTCGTATTACC-3′;
(c) siSphk2:
  (SEQ ID NO: 3)
5′-CAGGATTGCGCTCGCTTTCAT-3′;
(d) siRunx2:
  (SEQ ID NO: 4)
5′-CAGAAGAATGGTACAAATCCAAG-3′;
(e) sieIF3a:
  (SEQ ID NO: 5)
5′-GCAGATGGTCTTAGATATA-3′;
(f) SiEDB:
  (SEQ ID NO: 6)
5′-AAGGTATCCCTATTTTTGAAGCCTGTCTC-3′;
(g) siRe1A:
  (SEQ ID NO: 7)
5′-GGU GCA GAA AGA CAU UdTdT-3′;
(h) SiIL-10:
  (SEQ ID NO: 8)
5′-UUUAGCUACUACCGCAGCGdTdT-3′;
(i) siGPNMB:
  (SEQ ID NO: 9)
5′-GCACGGGUUUCUAUAAACAdTdT-3′;
(j) siSPARC:
  (SEQ ID NO: 10)
5′-AACAAGACCUUCGACUCUUCC-3′.
```

14. The dissolvable microneedle patch according to Clause 13, wherein the siRNA is siSPARC: 5′-AACAAGACCUUCGACUCUUCC-3′ (SEQ ID NO:10).

15. The dissolvable microneedle patch according to any one of the preceding clauses, wherein the plurality of microneedle structures extending from the substrate portion have a height of from 100 μm to 2.5 mm, such as from 200 μm to 1.5 mm, such as from 300 μm to 500 μm.

16. Use of a dissolvable microneedle patch according to any one of Clauses 1 to 15 in medicine.

17. Use of a dissolvable microneedle patch according to any one of Clauses 1 to 15 for the manufacture of a medicament for the treatment of a skin condition or disorder.

18. A dissolvable microneedle patch according to any one of Clauses 1 to 15 for use in the treatment of a skin condition or disorder.

19. A method of treatment of a skin disorder or condition, which method comprises the administration of a dissolvable microneedle patch according to any one of Clauses 1 to 15.

20. The use according to Clause 17, the use according to Clause 18 and the method according to Clause 19, wherein the skin condition or disorder is selected from one or more of the group consisting of scarring (e.g. hypertrophic scarring), keloid formation, eczema, and hyperpigmentation.

21. The uses and the method according to Clause 20, wherein the skin condition or disorder is hypertrophic scarring.

22. The uses and the method according to Clause 21, wherein the dissolvable microneedle patch is applied to the skin after wound union has occurred, optionally wherein the dissolvable microneedle patch is applied to the skin about two weeks after the wound was created.

### DRAWINGS

FIG. 1 depicts the schematic of gelatin (Gtn) conjugated with tyramine chloride (Tyr) to (a) create a net positive charge environment for (b) formation of nanoplexes with negatively charged siSPARC. The formation of Gtn-Tyr/siSPARC nanoplexes enhances the cell internalization and gene knockdown. The Gtn-Tyr/siSPARC is loaded onto the microneedle tip for transdermal siRNA delivery, demonstrated in scar prevention application.

FIG. 2 depicts (a) representative images of human dermal fibroblasts (HDFs) without treatment, incubated with 4 nmol/mL naked FAM-siSPARC, and 4 nmol/mg FAM-siSPARC/Gtn-Tyr (left to right). Scale bar represents 100 μm; (b) graph showing zeta potential of Gtn-Tyr with +1.77±0.18 mV was significantly reduced (P<0.0001) to +1.35±0.09 mV after interaction with 4 nmol/mL siSPARC. **** denote P<0.0001; and (c) average size (d.nm) of 4 samples of Gtn-Tyr prepared using different conditions after 1-day treatment using 1.0 units/mL collagenase type I.

FIG. 3 depicts the size measurement of Gtn-Tyr/siSPARC nanoplexes, comparing Z-average (d.nm) with Gtn and Gtn-Tyr.

FIG. 4 depicts the SPARC expression of HDFs at day 3 after treatment with 4 nmol naked siSPARC and 4 nmol Gtn-Tyr/siSPARC nanoplexes as compared to non-treated cells. * denotes P<0.05, * denotes P<0.01 and **** denotes P<0.0001.

FIG. 5 depicts the SPARC expression of HDFs at day 2 after treatment with nanoplexes formed between 4 nmol siSPARC and G1 (−1.7±0.3 mV), G2 (−0.6±0.3 mV) or G3 (+7.8±0.4 mV) as compared to control containing G1 and 4 nmol siScramble nanoplexes. * denotes P<0.01 and ** denotes P<0.0001.

FIG. 6 depicts (A) dissolvable microneedles containing siRNA-Gtn nanocomplex (Gtn-Tyr/siSPARC nanoplex); and (B) the simplified steps of making dissolvable microneedles containing siRNA-Gtn nanocomplex (Gtn-Tyr/siSPARC nanoplex).

FIG. 7 depicts (a) stereomicroscopic image showing pyramidal hyaluronic acid (HA) microneedles (Scale bar represents 500 μm); and (b) fluorescence microscopic image showing the HA needle tip concentrated with fluorescence tagged siSPARC (Scale bar represents 100 μm). The length of the current microneedle is 500 μm.

FIG. 8 depicts the gross view of wounds with no treatment and post-treated with blank, siScramble-loaded and siSPARC-loaded HA microneedles at day 8. Scale bar represents 1 mm.

FIG. 9 depicts (a) the percentage of wound area from day 4 to 8 comparing non-treated (NT) control with microneedles treatment. Wound area was normalized with wound area at day 4 (time-point for wound re-epithelialization and microneedle treatment); and (b) percentage healed area of mice's skin wound with different microneedles treatment as compared to NT control.

FIG. 10 depicts (a) Hematoxylin and Eosin (H&E) staining and Picro Sirius Red (PSR) staining of mice's skin wound at day 8 post-treated with siSPARC microneedles, comparing with NT wound, blank microneedles and siScramble loaded microneedles as controls (Scale bar represents 200 μm); and (b) percentage of collagen of mice's skin wound at day 8 post-treated with siSPARC loaded microneedles (* denotes P<0.05 and ** denotes P<0.01).

FIG. 11 depicts the analysis of immune response after microneedle treatment in mice's skin wound. Flow cytometric analysis results showing percentages of infiltrated CD11b⁺ myeloid-cells (lower right region) and CD11b⁺Lys6G⁺ neutrophils (upper right region) in mice's skin wound at day 8 after treatment with (a) blank microneedles, and microneedles loaded with (b) 4 nmol siScramble; and (c) 4 nmol siSPARC, as compared to (d) no treatment. Unstained cells (e) were used for gating purpose.

FIG. 12 depicts the analysis of immune response after microneedle treatment in mice's skin wound. Flow cytometric analysis results showing percentages of infiltrated CD11b⁺ myeloid-cells (lower right region) and CD11b⁺F4/80⁺ macrophages (upper right region) in mice's skin wound at day 8 after treatment with (a) blank microneedles, and microneedles loaded with (b) 4 nmol siScramble; and (c) 4 nmol siSPARC, as compared to (d) no treatment. Unstained cells (e) were used for gating purpose.

FIG. 13 depicts the gene expression of SPARC normalized against NT wound and housekeeping gene TBP for the mice's skin wound treated with siSPARC microneedles at day 8 (**** denotes P<0.0001).

FIG. 14 depicts the siRNA-embedded dissolving microneedles on a hard plastic polymer base with a cap.

FIG. 15 depicts the close-up photo of the microneedles on a hard plastic polymer base.

FIG. 16 depicts the flexible siRNA-embedded dissolving microneedle patches.

FIG. 17 depicts the structure of the microneedles in the flexible patch. The shaded portion comprises a contiguous layer of siRNA and HA.

FIG. 18 depicts the close-up photo of the microneedles in the flexible patch.

FIG. 19 depicts the microneedle patch after 3 hours of application on human skin, with dissolution of about half of the needles.

FIG. 20 depicts the microneedle patch after 6 h of application on human skin, with complete dissolution of the needles in most areas.

FIG. 21 depicts the after application of the hard-base patch for 5 min. The HA microneedles dissolved in the skin and a regular array of microneedles can be visualized (one being in the cross hair). This en face (facing forward) image (horizontal section) was taken at the level of the mid-dermis.

FIG. 22 depicts the after application of the flexible patch for 10 min. HA microneedles dissolved in the epidermis can be visualized (circled).

FIG. 23 depicts the surgical wound at day 14, just before application of the siRNA microneedle patch.

FIG. 24 depicts one month after application of the hard-base siRNA microneedle patch once every 3 days. The wound was healing well.

### DESCRIPTION

It has been surprisingly found that the current platform technology demonstrated effectiveness as a topical targeted SPARC gene therapy for collagen downregulation and wound closure, and is a promising and safe scar treatment method.

Thus, in a first aspect of the invention, there is provided a dissolvable microneedle patch, comprising:
a substrate portion comprising a first biocompatible material; and
a plurality of microneedle structures extending from the substrate portion, where each microneedle structure comprises:
   a base portion comprising a second biocompatible material; and
   a tip portion, wherein the tip portion comprises:
      a nanoplex formed from a positively charged biopolymer that incorporates a cell targeting moiety and an siRNA; and
      a third biocompatible material.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions.

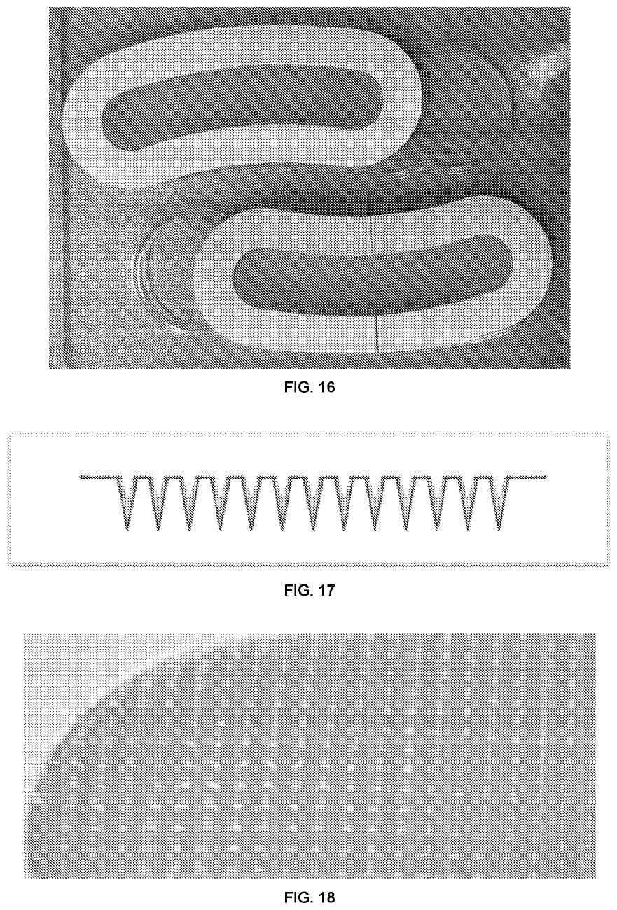

When used herein, the term “microneedle patch” refers to any dosage form that includes a substrate portion comprising a biocompatible material (polymer) from which a plurality of microneedle structures extend. For example, the microneedle patch may be fitted to a hard stamp (e.g. see the structure of FIG. 15) or the microneedle patch may include a peripheral region that does not have any needles, but which is coated with (or has itself) adhesive properties (e.g. see FIG. 16). Any other suitable form of the microneedle patch may be used herein.

The microneedles formed herein may include a base portion and a tip portion. The base portion is the part of the microneedles that extends from the substrate portion and the tip portion extends from the base portion to provide a pointed end to the microneedle.

The current invention is intended to pierce through at least the stratum corneum into the epidermis or it may pierce through the stratum corneum and the epidermis into the dermis. As such, the plurality of microneedle structures (or simply “microneedles”) extending from the substrate portion may have any suitable height that achieves these goals. For example, the plurality of microneedle structures extending from the substrate portion may have a height of from 100 μm to 2.5 mm, such as from 200 μm to 1.5 mm, such as from 300 μm to 500 μm. For the avoidance of doubt, the height of the microneedle structures is measured from the start of the base portion on the substrate's surface to the tip of the microneedle.

For the avoidance of doubt, it is explicitly contemplated that where a number of numerical ranges related to the same feature are cited herein, the end points for each range are intended to be combined in any order to provide further contemplated (and implicitly disclosed) ranges.

Thus, it is explicitly contemplated that the plurality of microneedle structures extending from the substrate portion may have a height of:
from 100 μm to 200 μm, from 100 μm to 300 μm, from 100 μm to 500 μm, from 100 μm to 1.5 mm, from 100 μm to 2.5 mm;
from 200 μm to 300 μm, from 200 μm to 500 μm, from 200 μm to 1.5 mm, from 200 μm to 2.5 mm;
from 300 μm to 500 μm, from 300 μm to 1.5 mm, from 300 μm to 2.5 mm;
from 500 μm to 1.5 mm, from 500 μm to 2.5 mm; and
from 1.5 mm to 2.5 mm.

In some embodiments, the dissolvable microneedle patch may comprise a plurality of microneedle structures with a tip portion, wherein the tip portion constitutes about 5% to about 99% of the total volume of the microneedle (i.e. the total volume of the tip and base portions). In some embodiments, the microneedle tip portion constitutes about 20% to about 90% of the total volume of the microneedle. In some embodiments, the microneedle tip portion constitutes about 50% of the total volume of the microneedle. In some embodiments, the microneedle tip portion constitutes about 75% of the total volume of the microneedle. In some embodiments, the microneedle tip portion constitutes about 5% to about 99%, about 5% to about 95%, about 5% to about 90%, about 5% to about 85%, about 5% to about 80%, about 5% to about 75%, about 5% to about 70%, about 5% to about 60%, about 5% to about 50%, about 5% to about 40%, about 5% to about 30%, about 5% to about 20%, about 5% to about 15%, about 5% to about 10%, about 10% to about 99%, about 10% to about 95%, about 10% to about 90%, about 10% to about 85%, about 10% to about 80%, about 10% to about 75%, about 10% to about 70%, about 10% to about 60%, about 10% to about 50%, about 10% to about 40%, about 10% to about 30%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about 15% to about 60%, about 15% to about 50%, about 15% to about 40%, about 15% to about 30%, about 15% to about 20%, about 25% to about 99%, about 25% to about 95%, about 25% to about 90%, about 25% to about 85%, about 25% to about 80%, about 25% to about 75%, about 25% to about 70%, about 25% to about 60%, about 25% to about 50%, about 25% to about 40%, about 25% to about 30%, about 50% to about 99%, about 50% to about 95%, about 50% to about 90%, about 50% to about 85%, about 50% to about 80%, about 50% to about 75%, about 50% to about 70%, about 50% to about 60%, about 60% to about 99%, about 60% to about 95%, about 60% to about 90%, about 60% to about 85%, about 60% to about 80%, about 60% to about 75%, about 60% to about 70%, about 70% to about 99%, about 70% to about 95%, about 70% to about 90%, about 70% to about 85%, about 70% to about 80%, about 70% to about 75%, about 80% to about 99%, about 80% to about 95%, about 80% to about 90%, about 80% to about 85%, about 90% to about 99%, about 90% to about 95%, or a value within these ranges. Specific examples may include about 99%, about 95%, about 90%, about 85%, about 80%, about 75%, about 70%, about 65%, about 60%, about 55%, about 50%, about 45%, about 40%, about 35%, about 30%, about 25%, about 20% about 15%, about 10%, about 9%, about 8%, about 7%, about 6%, about 5%, or a range between any of these values.

The plurality of microneedle structures may comprise one or more therapeutically active ingredients. The plurality of microneedle structures may each comprise the same therapeutically active ingredient. In some embodiments, the plurality of microneedle structures each comprise about the same amount of the therapeutically active ingredient. In some embodiments, the plurality of microneedle structures each comprise different amounts of the therapeutically active ingredient. Alternatively, the plurality of microneedle structures may be split into two or more sets (e.g. two or three sets), where the microneedle structures in each set comprise the same therapeutically active ingredient, but the microneedle structures in different sets comprise different therapeutically active ingredients. In some embodiments, the plurality of microneedle structures in each set each comprise about the same amount of the therapeutically active ingredient. In some embodiments, the plurality of microneedle structures in each set each comprise different amounts of the therapeutically active ingredient.

The term “dissolvable” when used herein refers to the fact that at least part of the tip portion of the microneedle patch that penetrates the skin of a subject will dissolve over a period of time after being applied to a subject. For example, the period of time may be from 3 to 24 hours, such as from 4 to 10 hours, such as 10 hours to achieve substantially complete dissolution (i.e. from 95 to 100%) of the microneedles on the patch.

Each of the first, second and third biocompatible materials may be any suitable biocompatible material. For example, each of the first, second and third biocompatible materials may be independently selected from the group including, but not necessarily limited to, hyaluronic acid, other dissolvable and biocompatible polymers, and combinations thereof. These other dissolvable and biocompatible polymers may include, but are not necessarily limited to polysaccharides, cellulose derivatives, synthetic polymers, and combinations thereof.

Examples of polysaccharides that may be mentioned herein include, but are not limited to chitosan, sodium alginate, dextran, sodium chondroitin sulphate, and combinations thereof.

Examples of cellulose derivatives that may be mentioned herein include, but are not limited to carboxymethyl cellulose, hydroxypropyl cellulose, hydroxypropyl methylcellulose, and combinations thereof.

Examples of synthetic polymers that may be mentioned herein include, but are not limited to, polyvinyl alcohol, polyvinylpyrrolidone, methylvinylether-co-maleic anhydride, and combinations thereof.

As noted above, the first, second and third biocompatible materials may be the same or different. For example:
the first, second and third biocompatible materials may all be different biocompatible materials; the first and the second biocompatible materials may be the same, while the third biocompatible material is different;
the first and the third biocompatible materials may be the same, while the second biocompatible material is different;
the second and the third biocompatible materials may be the same, while the first biocompatible material is different;
the first, second and third biocompatible materials may all be the same biocompatible material.

In embodiments of the invention that may be mentioned herein, the third biocompatible material may be hyaluronic acid. Additionally, the first and the second biocompatible materials may comprise (or be) hyaluronic acid.

As will be appreciated, the current invention requires the presence of a plurality of nanoplexes in conjunction with a biocompatible material (i.e. the third biocompatible material). A “nanoplex” when used herein refers to a complex having a diameter of from 1 to 999 nm that is formed by a therapeutic agent (e.g. a drug molecule) with a polymeric material that has an overall charge opposite to that of the therapeutic agent. Both cationic and anionic therapeutics form complexes with oppositely charged polymeric materials. Thus, in the current invention, the nanoplex may be formed from a positively charged biopolymer and an siRNA as the active therapeutic agent.

It is noted that the biopolymer in the tip portion incorporates a cell targeting moiety. This may be an inherent feature of the biopolymer (e.g. a feature of gelatin) or it may be a foreign feature introduced to the biopolymer/biomolecule by covalently bonding a molecule (e.g. a peptide or protein) to the biopolymer. Suitable cel targeting moiety is selected from one or more of gelatin, transferrin (targeting immune cells for skin inflammation diseases; see Xie, Yuran, et al. “Targeted delivery of siRNA to activated T cells via transferrin-polyethylenimine (Tf-PEI) as a potential therapy of asthma.” Journal of Controlled Release 229 (2016): 120-129), cyclic-Arginine-Glycine-Aspartic acid (RGD) tripeptide (targeting integrins including αᵥβ₃ for skin melanoma; Sebak, Aya Ahmed, et al. “Targeted photodynamic-induced singlet oxygen production by peptide-conjugated biodegradable nanoparticles for treatment of skin melanoma.” Photodiagnosis and photodynamic therapy 23 (2018): 181-189), skin protein-derived peptides (e.g. one or more of the group consisting of KTTKS (SEQ ID NO: 11), NAP-amide, and Lam332; e.g. see Cho, Jung Hyeon, et al. “Skin protein-derived peptide-conjugated vesicular nanocargos for selected skin cell targeting and consequent activation.” Journal of Materials Chemistry B (2021)), and folic acid (targeting skin melanoma cells; e.g. see Nawaz, Asif, and Tin Wui Wong. “Chitosan-carboxymethyl-5-fluorouracil-folate conjugate particles: microwave modulated uptake by skin and melanoma cells.” Journal of Investigative Dermatology 138.11 (2018): 2412-2422). The conjugation between the biopolymer/biomolecule (e.g. gelatin) and the abovementioned foreign targeting moieties can be formed through established carbodiimide crosslinking reaction, forming an amide bond.

As noted above, the nanoplex may comprise or consist of a positively charged biopolymer that incorporates a cell targeting moiety and an siRNA. Any suitable positively charged polymer that is biocompatible and dissolvable may be used herein. For example, the positively charged biopolymer may be formed from:
a heterobifunctional polymer comprising a first set of functional groups that are positively charged and a second set of functional groups that are negatively charged in an aqueous environment; and
a molecule conjugated to the heterobifunctional polymer, where the molecule is conjugated by a functional group that forms a bond with the second set of functional groups of the heterobifunctional polymer, so as to reduce the number of the second functional groups in the second set of functional groups, thereby providing a positively charged biopolymer.

The heterobifunctional polymers used herein are materials that contain two (i.e. a first and a second) oppositely charged sets of functional group types when placed in an aqueous environment. When discussed herein, the first set of functional groups are positively charged and the second set of functional groups are negatively charged. While a heterobifunctional polymer may naturally have an overall neutral charge (i.e. it is essentially zwitterionic) or it may have an overall negative charge, when it is used herein it has an overall positive charge.

When used herein, the term “overall charge” is used to refer to the net charge of the material being discussed. Suitable heterobifunctional polymers include, but are not limited to gelatin, collagen, silk fibroin, elastin, H₂N-PEG-CO₂H and combinations thereof. In particular embodiments that may be mentioned herein, the heterobifunctional polymer may be gelatin.

The aqueous environment referred to above may be any suitable aqueous environment, but may be particularly ones having a pH value of from 5 to 8, such as from 6.4 to 7.5, such as from 7.35-7.45. For the avoidance of doubt, when a number of different numerical ranges are provided, all possible combinations of the numerical point values provided to create further ranges are explicitly contemplated within the invention. For example, the ranges above provide the following pH ranges:
from 5 to 6.4, from 5 to 7.35, from 5 to 7.45, from 5 to 7.5, from 5 to 8;
from 6.4 to 7.35, from 6.4 to 7.45, from 6.4 to 7.5, from 6.4 to 8;
from 7.35 to 7.45, from 7.35 to 7.5, from 7.35 to 8;
from 7.45 to 7.5, from 7.45 to 8; and from 7.5 to 8.

As will be appreciated, the term “PEG” used herein is an abbreviation of polyethylene glycol. Moreover, it is used herein as a banner term to cover polyethylene glycol and polyethylene oxide, which are both terms for a polymer/oligomer of the formula H—(O—CH₂—CH₂)ₙ—OH, but which differ on molecular weight. The term PEG when used herein may refer to a polymeric material having the formula H—(O—CH₂—CH₂)ₙ—OH with a molecular mass below 20,000 g/mol, based on the number average molecular weight. The term PEO when used herein may refer to a polymeric material having the formula H—(O—CH₂—CH₂)ₙ—OH with a molecular mass above 20,000 g/mol, based on the number average molecular weight.

The molecule conjugated to the heterobifunctional polymer forms a covalent bond with the negatively charged functional group in the second set of functional groups, thereby removing negative charges from the polymer, resulting in the polymer having an overall positive charge. As will be appreciated, some or all of the negatively charged functional groups may be conjugated, such that the overall charge of the resulting material is modified. The portion of the second set of functional groups that is conjugated may be from 0.01% to 100% of the second set of functional groups on the heterobifunctional polymer. For example, even if the unmodified heterobifunctional polymer has an overall negative charge, the reaction with the molecule that conjugates the negatively charged second set of functional groups may cause the material to end up with an overall positive charge. Thus, in this example, the molecule is selected to have a functional group that can react with the second set of functional groups on the heterobifunctional polymer, thereby reducing the negative charges present in the polymeric backbone and thereby changing the overall charge of the resulting material. The extent of the resulting overall positive charge depends on the amount of the molecule used relative to the amount of the second set of functional groups on the heterobifunctional polymer.

As noted, the molecule contains a functional group that is suitable to form a bond with the functional group in the second set of functional groups. The functional group in the molecule may be selected from one or more of amide, amino and ammonium (it will be appreciated that amino and ammonium may be effectively the same substance).

As will be appreciated, the functional group in the molecule that is suitable to form a bond with the second set of functional groups (of the heterobifunctional polymer) may itself be a functional group that has the opposite polarity (i.e. is positively charged). For example, the functional group suitable to form a bond with the second set of functional groups of the heterobifunctional polymer may be positively charged in an aqueous environment (e.g. at the pH ranges mentioned above) and may be selected from an amino group. For the avoidance of doubt, reference to an amino group in the context of forming a bond with the second set of functional groups refers to an amino functional group capable of forming a covalent bond and so it excludes quaternary and tertiary amino groups.

Given the above, the molecule conjugated to the heterobifunctional polymer may be selected from one or more of 4-hydroxybenzylamine, dopamine, and tyramine. In embodiments that may be mentioned herein, the molecule conjugated to the heterobifunctional polymer may be tyramine.

As noted above, the nanoplexes disclosed herein make use of an siRNA as the active therapeutic agent. Any suitable siRNA with a therapeutic use, whether systemic or local, may be used herein. For example, the siRNA may be selected from one or more of the group consisting of:

```
(a) SITGFβ1-337:
  (SEQ ID NO: 1)
5′-GGUGGAAACCCACAACGAATT-3′;
(b) SiFAK:
(SEQ ID NO: 2)
5′-GCGAAATCCA-TAGCAGGCCACTATAGTGAGTCGTATTACC-3′;
(c) siSphk2:
  (SEQ ID NO: 3)
5′-CAGGATTGCGCTCGCTTTCAT-3′;
(d) siRunx2:
  (SEQ ID NO: 4)
5′-CAGAAGAATGGTACAAATCCAAG-3′;
(e) sieIF3a:
  (SEQ ID NO: 5)
5′-GCAGATGGTCTTAGATATA-3′;
(f) SiEDB:
  (SEQ ID NO: 6)
5′-AAGGTATCCCTATTTTTGAAGCCTGTCTC-3′;
(g) siRe1A:
  (SEQ ID NO: 7)
5′-GGU GCA GAA AGA CAU UdTdT-3′;
(h) SiIL-10:
  (SEQ ID NO: 8)
5′-UUUAGCUACUACCGCAGCGdTdT-3′;
(i) SiGPNMB:
  (SEQ ID NO: 9)
5′-GCACGGGUUUCUAUAAACAdTdT-3′;
(j) siSPARC:
  (SEQ ID NO: 10)
5′-AACAAGACCUUCGACUCUUCC-3′.
```

As will be appreciated, the specific combination of the cell targeting moiety incorporated into the positively charged biopolymer and the siRNA may be chosen to ensure that the siRNA is directed towards the desired site of action.

For example, if one is targeting the treatment of fibroblasts in the dermis with one or more siRNAs, one may select a cell targeting moiety that will enable the siRNA to be directed towards the cell of interest. For example, gelatin alone may be suitable for this use, or the cell targeting moiety may be the skin protein-derived peptide KTTKS (SEQ ID NO: 11). As will be appreciated, the skilled person may prepare any suitable combination of the cell targeting moiety and siRNA based on the desired treatment and targeted cell.

Specific conditions that may be treated with the siRNAs disclosed above include, but are not limited to, the following.

### Hypertrophic Scarring

Targeted Region: Dermis
Targeted Cells: Fibroblasts

(i) siTGFβ1-337: 5′-GGUGGAAACCCACAACGAATT-3′ (SEQ ID NO:1) (Target TGFβ1, reduction of collagen content). Zhao, Rui, et al. “Transdermal siRNA-TGFβ1-337 patch for hypertrophic scar treatment.” Matrix Biology 32.5 (2013): 265-276.

(ii) siFAK: 5′-GCGAAATCCA-TAGCAGGCCACTATAGTGAGTCGTATTACC-3′ (SEQ ID NO: 2) (FAK was effectively blocked, accompanied by decreasing expression of integrin α, TGF-β and α-SMA, decreases amount of collagen synthesis). Chen, Rui, et al. “Focal adhesion kinase (FAK) siRNA inhibits human hypertrophic scar by suppressing integrin α, TGF-β and α—SMA.” Cell biology international 38.7 (2014): 803-808.

(iii) siSphk2: 5′-CAGGATTGCGCTCGCTTTCAT-3′ (SEQ ID NO:3) (Inhibition of human scar fibroblast proliferation, promotion of apoptosis, and inactivation of TGF-β1/Smad signaling and collagen I expression). Zeng, Jian, et al. “Inhibition of sphingosine kinase 2 attenuates hypertrophic scar formation via upregulation of Smad7 in human hypertrophic scar fibroblasts.” Molecular Medicine Reports 22.3 (2020): 2573-2582.

### Keloids

Targeted region: Dermis
Targeted cells: Fibroblasts

(i) siRunx2: 5′-CAGAAGAATGGTACAAATCCAAG-3′ (SEQ ID NO: 4) (Inhibited the human keloid fibroblast proliferation, migration, expression levels of ECM-related proteins through the suppression of the PI3K/AKT signalling pathway). Lv, Wenchang, et al. “Treatment of keloids through Runx2 siRNA-induced inhibition of the PI3K/AKT signalling pathway.” Molecular medicine reports 23.1 (2020): 1-1.

(ii) sieIF3a: 5′-GCAGATGGTCTTAGATATA-3′ (SEQ ID NO:5) (Inhibits ECM expression in keloid fibroblasts). Li, Tianyu, and Junxiang Zhao. “Knockdown of eIF3a inhibits TGF-β1-induced extracellular matrix protein expression in keloid fibroblasts.” Molecular Medicine Reports 17.3 (2018): 4057-4061.

(iii) siEDB: 5′-AAGGTATCCCTATTTTTGAAGCCTGTCTC-3′ (SEQ ID NO:6) (Fibronectin extra domain B silencing can inhibit TGF-β1-induced cell proliferation and collagen deposition in keloid fibroblasts). Cui, Jingbo, et al. “Knockdown of fibronectin extra domain B suppresses TGF-β1-mediated cell proliferation and collagen deposition in keloid fibroblasts via AKT/ERK signaling pathway.” Biochemical and Biophysical Research Communications (2020).

### Eczema

Targeted region: Dermis
Targeted cells: CD4+ (Th2 helper cells)

(i) siRelA: 5′-GGU GCA GAA AGA CAU UdTdT-3′ (SEQ ID NO:7) (Inhibited RelA and inflammatory cytokine production). Ibaraki, Hisako, et al. “Transdermal anti-nuclear kappaB siRNA therapy for atopic dermatitis using a combination of two kinds of functional oligopeptide.” International journal of pharmaceutics 542.1-2 (2018): 213-220.

(ii) siIL-10: 5′-UUUAGCUACUACCGCAGCGdTdT-3′ (SEQ ID NO:8) (Blocking of endogenous cytokine). Kigasawa, K., et al. “Noninvasive delivery of siRNA into the epidermis by iontophoresis using an atopic dermatitis-like model rat.” International journal of pharmaceutics 383.1-2 (2010): 157-160.

### Hyperpigmentation

Targeted region: Epidermis
Targeted cells: Melanocytes

siGPNMB: 5′-GCACGGGUUUCUAUAAACAdTdT-3′ (SEQ ID NO:9) (Silencing of glycoprotein non-metastatic melanoma protein). Shi, Fangyuan, et al. “Induction of matrix metalloproteinase-3 (MMP-3) expression in the microglia by lipopolysaccharide (LPS) via upregulation of glycoprotein nonmetastatic melanoma B (GPNMB) expression.” Journal of Molecular Neuroscience 54.2 (2014): 234-242.

In embodiments of the invention, the siRNA may be siSPARC.

The microneedles may contain one or more of the siRNAs above. That is, the siRNAs may be used alone or in combination. This combination may be in the same tip (e.g. when being targeted at the same cell) or they may be in different sets of tips, thereby allowing more than one cell and/or condition to be targeted for treatment at the same time.

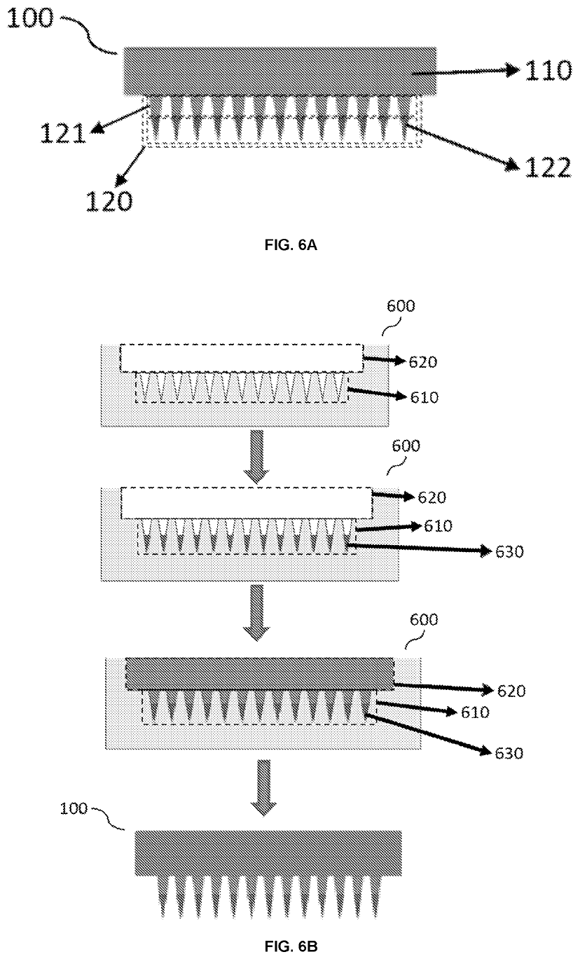

As depicted in FIG. 6A, the dissolvable microneedle patch 100 includes a substrate portion 110, a plurality of microneedle structures extending from the substrate portion 120 comprising a base portion 121 and a tip portion 122. The drugs/active therapeutic agent(s) can be loaded within the microneedles' polymer matrix, thus increasing their drug loading capacity in one convenient formulation. The dissolvable microneedle patches disclosed herein may be formed using a simple process as set out in FIG. 6B.

In the first step of the process, a microneedle mold 600 is provided, which has a microneedle formation region 610 and a substrate formation region 620. A mixture 630 comprising the third biocompatible material and the nanoplex is poured into the microneedle formation region 610. As depicted, this mixture 630 will not fill up the entire volume of the microneedle formation region 610, so as to leave space for a base portion to be present. As will be appreciated, the addition of the mixture 630 will not be fully accurate and, as the process (described in more detail in the experimental section below) makes use of centrifugation, it is expected that a small portion of the mixture will end up adhered to the non-filled regions of the mold, such as the non-filled portion of the microneedle formation region 610 and the substrate formation region 620.

If the base portion of the microneedle and the substrate are to be formed of the same biocompatible material (i.e. the first and second biocompatible materials are the same material), then the remaining space in the microneedle formation region 610 and the entirety of the substrate formation region 620 may be filled by said material, which will result in the desired product 100. In the event that the second biocompatible material is different from the first biocompatible material, then it will be cast first and allowed to set (again, potentially leaving trace amounts of this second biocompatible material in the substrate formation region 620) before the first biocompatible material is added to the substrate formation region 620 and allowed to set to provide the product 100. Further details of the method used to make the embodiments disclosed herein are provided in the experimental section below.

The dissolvable microneedle patch may be utilised in a method of medical treatment. Thus, according to further aspects of the invention, there is provided:
(a) a dissolvable microneedle patch for use in medicine;
(b) a dissolvable microneedle patch for use in the manufacture of a medicament for the treatment of a skin condition or disorder;
(c) a dissolvable microneedle patch for use in the treatment of a skin condition or disorder; and
(d) a method of treatment of a skin disorder or condition, which method comprises the administration of a dissolvable microneedle patch.

It will be appreciated that any of the dissolvable microneedle patches disclosed herein may be used in the above applications.

The term “skin disorder or condition” will be understood by those skilled in the art to include scarring (e.g. hypertrophic scarring), keloid formation, eczema, and hyperpigmentation.

A particular skin disorder or condition that may be mentioned herein is hypertrophic scarring. When used for hypertophic scarring, the siRNA may be selected from one of these indicated herein to treat said condition and/or is siSPARC. This may be used in a dissolvable microneedle patch where the first to third biocompatible materials are hyaluronic acid and the nanoplex is completed by gelatin that is conjugated to tyramine. In any event, when seeking to treat hypertophic scarring, the timing of the application of the dissolvable microneedle patch may be chosen to minimise the possibility of scarring. For example, the dissolvable microneedle patch may be applied to a subject's skin after wound union has occurred. More particularly, the dissolvable microneedle patch may be applied to a subject's skin around or slightly around the time that collagen synthesis starts. If the dissolvable microneedle patch is applied too early, the dissolvable microneedle patch may inhibit wound healing. On the other hand, if the dissolvable microneedle patch is applied too late, the dissolvable microneedle patch may not prevent the formation of collagen bundles. For example, the dissolvable microneedle patch may be applied to the patient's skin about two weeks after the wound was created. It has been found that the application of the dissolvable microneedle patch two weeks after the wound was created grants good wound recovery, and subjects observed no side effects. It is noted that the first phase of wound healing is the inflammatory phase, which lasts from four to six days after the wound was created. The second phase, the proliferative phase, occurs from four to twenty-four days after the wound was created. During the proliferative phase, fibroblasts lay collagen, wound edges begin to contract, and epidermal cells migrate from the wound margins. Therefore, without wishing to be bound by theory, it is believed that the middle of the proliferative phase, at fourteen days (or two weeks) after the wound was created, is a good time to initiate the treatment discussed herein. However, it will be appreciated that the exact timing may be left to the hands of a skilled physician, depending on the subject in question.

Therefore, as will also be demonstrated in the examples, the dissolvable microneedle patch disclosed herein can achieve simultaneous drug penetration of skin barrier, delivery to a specific region or depth of the skin where the targeted cells reside, and finally, achieve targeting of the desired cells for effective siRNA silencing.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject The term does not denote a particular age or sex. Thus, adult and newborn subjects, whether male or female, are intended to be covered.

The term “effective amount” refers to an amount of a compound which confers a therapeutic effect on the treated patient (e.g. sufficient to treat or prevent the disease). The effect may be objective (i.e. measurable by some test or marker) or subjective (i.e. the subject gives an indication of or feels an effect).

The amount of the siRNA(s) present in the dissolvable microneedle patch used in accordance with the present invention will depend on various factors, such as the severity of the condition to be treated, the particular patient to be treated, as well as the compound(s) which is/are employed. In any event, the amount of siRNA used in the dissolvable microneedle patch may be determined routinely by the skilled person. For example, the tips of the dissolvable microneedle patches may contain from 0.1 to 99 wt % of the nanoplex, with the balance being the biocompatible material. In particular embodiments that may be mentioned herein, the ratio (wt/wt) of the siRNA to the biocompatible material may be from 0.001:1 to 0.1:1, such as from 0.007:1 to 0.08:1.

Depending on the disorder, and the patient, to be treated, the siRNA(s) may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent. The dosage may also be determined by the timing and frequency of administration. In the case of topical and/or parenteral administration the dosage can vary from about 0.00001 mg to about 10 mg per day, such as from 0.001 to 5 mg per day, of the siRNA(s).

In any event, the medical practitioner, or other skilled person, will be able to determine routinely the actual dosage, which will be most suitable for an individual patient. The abovementioned dosages are exemplary of the average case; there can, of course, be individual instances where higher or lower dosage ranges are merited, and such are within the scope of this invention.

Further aspects and embodiments of the invention will now be described by reference to the following non-limiting examples.

### EXAMPLES

### Materials

A 21-base double-stranded small interfering RNA for SPARC (siSPARC: 5′-AACAAGACCUUCGACUCUUCC-3′ (SEQ ID NO:10)) was used in the fabrication of microneedles and in vivo SPARC knockdown studies. A non-silencing scrambled control (siScramble: 5′-GCUCACAGCUCAAUCCUAAUC-3′ SEQ ID NO:12)) was also used. The siRNAs and FAM-siSPARC were synthesized and purified by Bioneer (Korea). Gelatin (Gtn) was purchased from Wako Pure Chemical Industries, Ltd., Japan. Tyramine chloride (Tyr.Cl), N-(3-Dimethylaminopropyl)-N′-ethylcarbodiimide hydrochloride (EDC·HCl), KAPA SYBR® FAST Universal, N-Hydroxysuccinimide (NHS) and Dulbecco's Modified Eagle Medium (DMEM) were purchased from Sigma-Aldrich, US. DNase-RNase-free distilled water, fetal bovine serum (FBS), antibiotic-antimycotic (ABAM) and Opti-MEM™ I reduced serum medium were purchased from Thermo Fisher Scientific, US. Polydimethylsiloxane (PDMS) and hyaluronic acid (HA) were provided by SMICNA Pte. Ltd., SG. Tegaderm® was purchased from 3M, US. PureLink™ RNA Mini was purchased from Invitrogen, Thermo Fisher Scientific, US. iScript cDNA synthesis kit was purchased from Bio-Rad, US. DEPC-treated water was provided by Bioneer, Korea.

### Analytical Techniques

### Steteomicroscopy

A ZEISS Stemi DV4 stereomicroscope (Germany) attached with a camera was used to take images of the Gtn-Tyr/siSPARC microneedle patch.

### Fluorescence Microscopy

A ZEISS Axio Observer Z1 fluorescence microscope (Germany) was used to perform fluorescence microscopy.

### Gtn-Tyr/siSPARC Nanoplexes' Size Measurement

The size of Gtn-Tyr/siSPARC nanoplexes were measured using the Malvern Zetasizer (Malvern Panalytical Ltd, UK). Freeze-dried Gtn-Tyr (1 mg) was dissolved in deionised (DI) water (0.4 mL) and allowed to electrostatic interact with 4 nmol siSPARC for 15 min. The size was measured in the disposable low volume cell at the temperature of 25° C. Each sample was tested three times, and the Z-average was recorded.

### Statistical Analysis

All data are expressed in mean±standard deviation with a replicate of n=3 unless otherwise specified. The differences between the values were assessed using one-way ANOVA, where P<0.05 was considered statistically significant. Differences are labelled * for P≤0.05, ** for P≤0.01, *** for P≤0.001 and **** for P≤0.0001.

### Example 1. Synthesis of Gtn-Tyr/siSPARC and FAM-siSPARC/Gtn-Tyr

### Gtn-Tyr/siSPARC Nanoplexes

Gtn was conjugated with a small phenol molecule, Tyr, using the common carbodiimide crosslinking reaction and previously established procedures by Wang et al. (Wang, L.-S. et al., Biomaterials 2010, 31, 8608-8616). Briefly, 2 w/v % of Gtn was dissolved in DI water by heating the solution at 60° C. Tyr.Cl (57.6 mM) was added to the solution. EDC·HCl (31.8 mM) and NHS (15.9 mM) were added into the reaction mixture to initiate the conjugation reaction. The reaction was allowed to proceed overnight at pH 4.7. After the reaction, the reaction mixture was purified by dialyzing against DI water for 2 days and finally, freeze-dried to obtain the Gtn-Tyr precursor. Next, 4 nmol/mg Gtn-Tyr/siSPARC was obtained by dissolving both lyophilized Gtn-Tyr and siSPARC in DEPC-treated water and allowed to electrostatic interact for 15 min to form the nanoplexes. The nanoplexes were then freeze-dried to obtain the powder form.

### Fam-siSPARC/Gtn-Tyr Nanoplexes

4 nmol/mg FAM-siSPARC/Gtn-Tyr nanoplex was prepared from Gtn-Tyr and FAM-siSPARC by following the protocol above. The nanoplexes were then freeze-dried to obtain the powder form.

### Example 2. Gtn-Tyr/siSPARC Nanoplexes for Effective siSPARC Protection, Cell Targeting and SPARC Gene Expression Knockdown

A safe and efficient siRNA protection and delivery to the cellular environment remains the critical bottleneck of siRNA therapeutics for clinical application. To achieve effective siRNA gene knockdown, the siRNA has to be (1) protected against endonucleases degradation and (2) successfully targeted onto the desired cells for cellular internalization.

### In Vitro Evaluation of Cell Initialization

HDFs were seeded with a cell density of 5×10⁴ cells/well on 24 well plate and cultured using DMEM high glucose supplemented with 10% FBS and 1% ABAM. Gtn-Tyr with positive surface charge (ZP=+6.29±0.43 mV) was used to electrostatic interact with siSPARC. Once the HDFs reached ˜80% confluency, the culture medium was removed and replaced with culture medium containing FAM-siSPARC (4 nmol/mL) or nanoplexes of FAM-siSPARC/Gtn-Tyr (4 nmol/mg). HDFs were incubated with the FAM-siSPARC or nanoplexes of FAM-siSPARC/Gtn-Tyr for 24 h at 37° C. and 5% CO₂. After the treatment, HDFs were stained with Hoechst33342 (Life Technologies, US), according to manufacturer's protocol. HDFs were then imaged using a ZEISS Axio Observer Z1 fluorescence microscope (ZEISS, Germany). All imaging settings were kept constant for any comparison between experimental conditions.

### Gtn-Tyr/siSPARC Nanoplexes-Assisted In Vitro SPARC Knockdown Study

HDFs were seeded onto a 12-well plate with a density of 6×10⁴ cells per well and cultured with 1 mL of DMEM high glucose supplemented with 10% FBS and 1% ABAM. HDFs were incubated at 37° C. and 5% CO₂. Once HDFs reached ˜80% confluency, the culture medium was replaced with 1 mL of Opti-MEM™ I reduced serum medium containing siSPARC (4 nmol) or 1 mg of Gtn-Tyr/siSPARC (4 nmol) nanoplexes. After being treated for 3 days, total RNA was recovered with PureLink™ RNA Mini, according to the manufacturer's recommendations. Total RNA extract (400 ng) was then converted to first-strand cDNA using iScript cDNA synthesis kit, according to the manufacturer's instructions. Quantitative real-time PCR (qPCR) was performed in 96-well microtiter plates (20 μL). Each reaction consisted of the 10 ng first-strand reaction product, 10 μM each of forward and reverse primers (Sigma-Aldrich, US), 10 μL of KAPA SYBR® FAST Universal, and 8.7 μL of DNase-RNase-free distilled water. Amplification and analysis of cDNA fragments were carried out using the CFX Connect Real-Time PCR System (Bio-Rad, US). All PCR reactions were performed in triplicate.

All mRNA levels were measured as cycle threshold (CT) levels and were normalized with the corresponding RPL13a CT values (housekeeping gene). Values are expressed as fold increase over the corresponding values for untreated cells by the 2^{-ΔΔCT} method.

### Results and Discussion

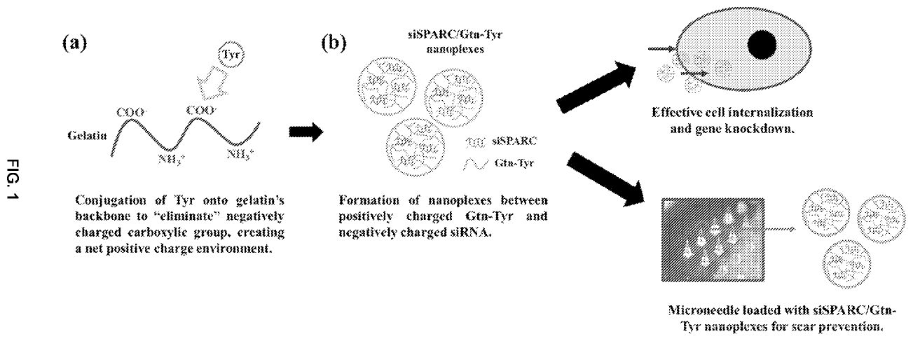

Gtn is a natural target for fibroblasts, which leads to clathrin-mediated endocytotic cellular internalization. As depicted in FIG. 1, when Gtn is conjugated with Tyr, the resultant Gtn (a) produces sufficient net positive charge for electrostatic interaction with net negative-charge siSPARC, leading to the formation of protective and cell-targeting nanoplexes to achieve effective cell internalization and gene knockdown effect.

Herein, the ability of Gtn-Tyr in protecting and enhancing siSPARC delivery into the cellular environment was demonstrated using HDFs (FIG. 2a). HDFs are involved in wound healing and an increase in SPARC production in HDFs was linked to fibrosis and scarring (Tracy, L. E., Minasian, R. A. & Caterson, E. J., Adv. Wound Care 2016, 5, 119-136). Comparing different treatment groups (FIG. 2a), HDFs treated with nanoplexes of FAM-siSPARC/Gtn-Tyr showed a significantly higher green fluorescence intensity as compared to Gtn-absent FAM-siSPARC. This observation supports that the positive-charge tuned Gtn, when electrostatically interacted with negative-charge siSPARC, was endocytosed by fibroblasts into the cytoplasm. The electrostatic interaction between the negatively charged siRNA and positive-charge tuned Gtn-Tyr was further verified by showing a significant reduction (P<0.0001) in overall zeta potential after the interaction between these two components (FIG. 2b). The current cell internalization study demonstrated the importance of using the positive-charge tuned Gtn-Tyr to form nanoplexes with siSPARC to enhance the protection and delivery of SPARC into the cellular environment. The siSPARC electrostatically bound to the positive-charge tuned Gtn-Tyr is subsequently internalized by the cells through a clathrin-mediated pathway.

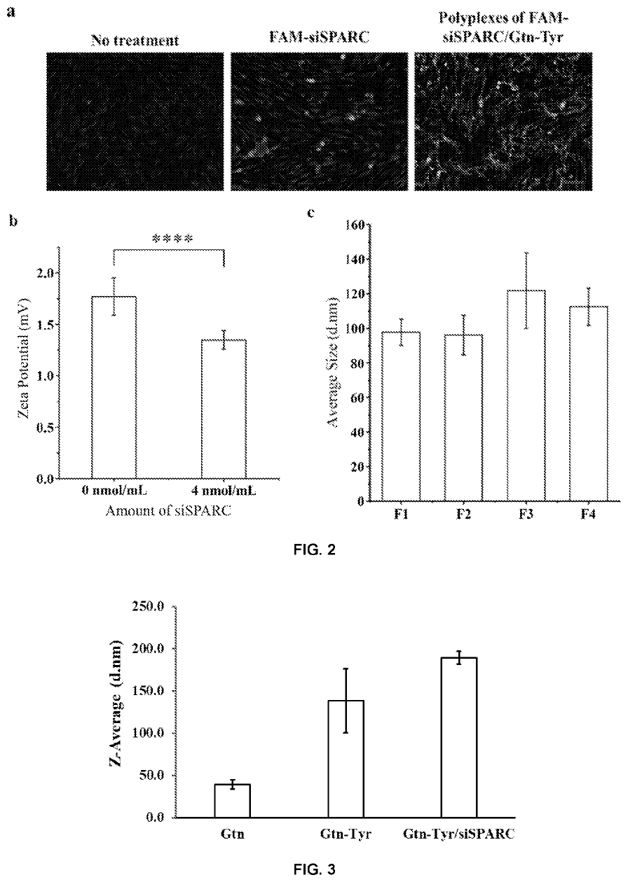

In this study, Gtn-Tyr with a surface potential of +7.2±0.2 mV was synthesized (Chun, Y. Y. et al., Sci. Rep. 2021, 11, 1470). Gtn without Tyr conjugation had a surface charge of −1.7±0.3 mV. Gtn-Tyr was then used to form nanoplexes with 4 nmol siSPARC. When comparing the size between samples (FIG. 3), Gtn without and with Tyr conjugation had a Z-average of 39.2±5.2 and 138.7±38.0 d.nm, respectively. When Gtn-Tyr formed nanoplexes with siSPARC, the sample had a Z-average of 189.6±7.7 d.nm. Thus, the Gtn-Tyr/siSPARC nanoplexes had an average size below the upper size limit (200 nm) of the clathrin-mediated endocytosis mechanism (McMahon, H. T. & Boucrot, E., Nat. Rev. Mol. Cell Biol. 2011, 12, 517; and Chun, Y. Y. et al., Sci. Rep. 2021, 11, 1470, FIG. 2c).

HDFs were treated with Gtn-Tyr/siSPARC for 3 days. Then, their SPARC expression was compared with HDFs without siSPARC treatment and treated with naked 4 nmol siSPARC.

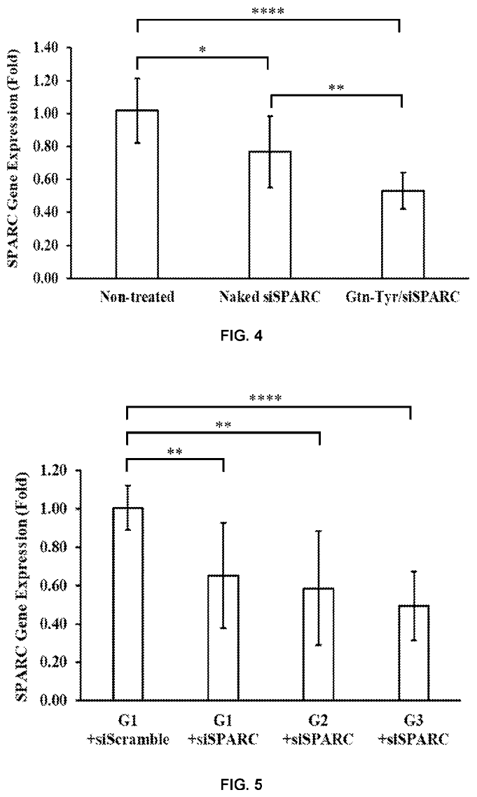

As shown in FIG. 4, there was a significant reduction in SPARC expression for naked siSPARC (0.77±0.22-fold, P<0.05) when compared to non-treated HDFs (1.02±0.20-fold). However, when treated with Gtn-Tyr/siSPARC using nanoplexes, the reduction of SPARC was significant (0.53±0.11-fold) as compared to both non-treated HDFs (P<0.0001) and HDFs treated with naked siSPARC (P<0.01). In conclusion, this study showed the importance of the Gtn-Tyr/siSPARC nanoplexes in enhancing cell internalization and gene knockdown efficiency. Further, the formation of Gtn-Tyr/siSPARC nanoplexes was demonstrated to achieve effective siSPARC protection and fibroblasts targeting, leading to effective cellular internalization and SPARC gene knockdown.

### Example 3. Effect of Gtn-Tyr's Surface Charge on the SPARC Knockdown Efficiency

In this study, the importance of controlling the amount of Tyr conjugated onto Gtn to control the surface charge of Gtn, which ultimately affects the efficiency of siSPARC cellular internalization and gene knockdown, was investigated.

Gtn-Tyr with Different Surface Charge in Vitro SPARC Knockdown Study

HDFs were cultured by following the SPARC knockdown protocol in Example 2. Gtn-Tyr with different surface charges (G1: −1.7±0.3 mV, no Tyr conjugated; G2: −0.6±0.3 mV; G3: +7.8±0.4 mV) were synthesized according to our previously established studies (Chun, Y. Y. et al., Sci. Rep. 2021, 11, 1470). 1 mg of Gtn sample was then used to form nanoplexes with 4 nmol siSPARC. The nanoplexes were then used to treat HDFs for 2 days. After being treated for 2 days, total RNA was recovered with PureLink™ RNA Mini (Invitrogen, Thermo Fisher Scientific, US) according to the manufacturer's recommendations. Total RNA extract (400 ng) was then converted to first-strand cDNA using iScript cDNA synthesis kit (Bio-Rad, US) according to manufacturer's instructions. qPCR and real-time PCR studies were performed by following the SPARC knockdown protocol in Example 2.

### Results and Discussion

Gtn conjugated with different amounts of Tyr (therefore with different zeta potential values) were prepared for comparison in this study, namely G1 with no Tyr conjugated (−1.7±0.3 mV), G2 with a net neutral charge (−0.6±0.3 mV), and G3 with net positively charge (+7.8±0.4 mV). The Gtn samples were then used to form nanoplexes with siSPARC (4 nmol) and treat HDFs for 2 days.

FIG. 5 shows an obvious decreasing SPARC expression (indicating an increasing SPARC knockdown efficiency) with increasing surface positive charge of Tyr-modified Gtn (G1 to G3) compared to the control (G1+4 nmol siScramble nanoplex). SPARC expression was significantly knockdown when treated with G1+siSPARC (0.65±0.27-fold, P<0.0001), G2+siSPARC (0.59±0.30-fold, P<0.01), and G3+siSPARC (0.49±0.18-fold, P<0.0001) compared to control G1+siScramble (1.01±0.11-fold).

Overall, this study showed the importance Tyr conjugation in Gtn to modify its surface charge to net positive charge for effective siSPARC protection, leading to enhanced cell internalization and gene knockdown. Thereafter, the Gtn-Tyr/siSPARC is loaded onto the microneedle tips to achieve effective transdermal siRNA delivery, and demonstrated in scar prevention in the following examples.

### Example 4. Fabrication of Gtn-Tyr/siSPARC-Loaded Microneedles

A pyramidal microneedle master structure was created using an electro-discharge machining process (Micropoint Technologies Pte Ltd, Singapore). Microneedle moulds were made from PDMS to inverse replicate the master structure, following a published procedure (Chen, M.-C. et al., Biomacmmolecules 2012, 13, 4022-4031). The PDMS moulds obtained were repeatedly used to make polymer microneedles. A two-step casting process was used to mould drug-loaded microneedle patches. Briefly, a mixture of Gtn/siSPARC nanoplexes (Gtn-Tyr/siSPARC nanoplex, 4 nmol/mg) and HA (0.3 g) in DI water (1 mL) was first cast and centrifuged to the needle tip positions of the mould. Excess solution on the mould surface was removed and saved for future use. Next, 200 mg of HA solution (300 g/L) without the nanoplexes was added into the mould as mentioned above, centrifuged, and formed the microneedles' base. The microneedles were dried and removed (FIG. 6B).

### Characterization

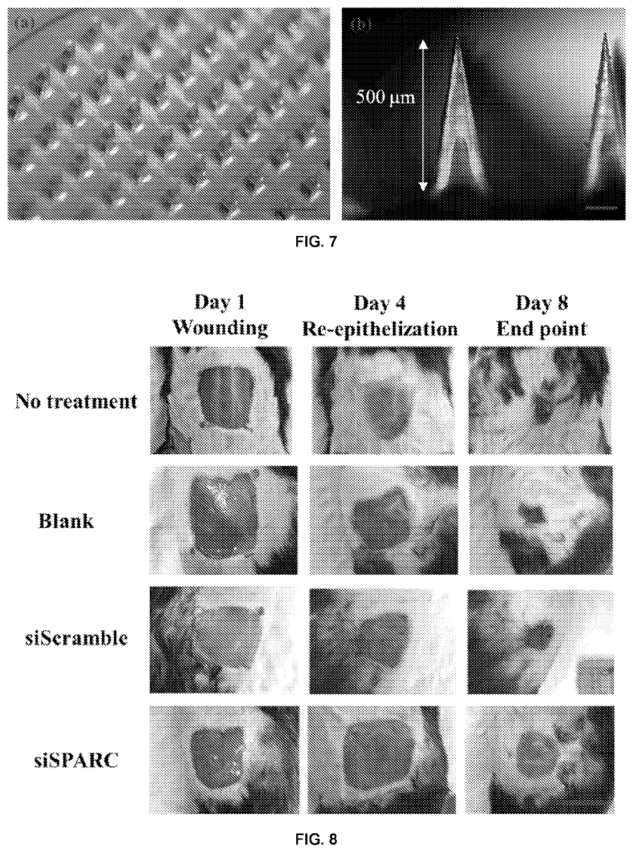

The microneedles were visualized under stereomicroscope and fluorescence microscope. FIG. 7a shows the pyramidal HA microneedles fabricated using the electro-discharge machining process, where drug/siRNA were shown to be loaded and concentrated at the tip of the microneedles (FIG. 7b). The length of the current microneedle is 500 μm, designed to target fibroblasts in the dermis.

### Example 5. Wound Observation and Closure Rate

### Wound Experiment

Wild type C57BL/6J male mice 8-9 weeks of age (NTU, ARF) were housed in a 12:12 light-dark cycle, temperature-controlled room (22±1° C.) and were fed with standard mouse chow diet and water ad libitum. Full-thickness excisional wounds of 1×1 cm were inflicted, as previously described (Tan, N. S. & Wahli, W., Curr. Protoc. Mouse Biol. 2013, 3, 171-185), on the mice and Tegaderm®. Upon wound re-epithelization on day 4, wounds were sterilized with alcohol swabs before microneedles application of treatment arms, where microneedles were applied with pressure for 1 min on the wounds, or control treatment with no needles applied, before the reapplication of Tegaderm®. Mice were then sacrificed on day 8, and the wounds were collected and subjected to downstream histological, flow cytometry and hydroxyproline assay examinations that will be discussed in Examples 6-8. All animal experiments were carried out following the guidelines provided by the institutional animal care and use committee.

### Results and Discussion

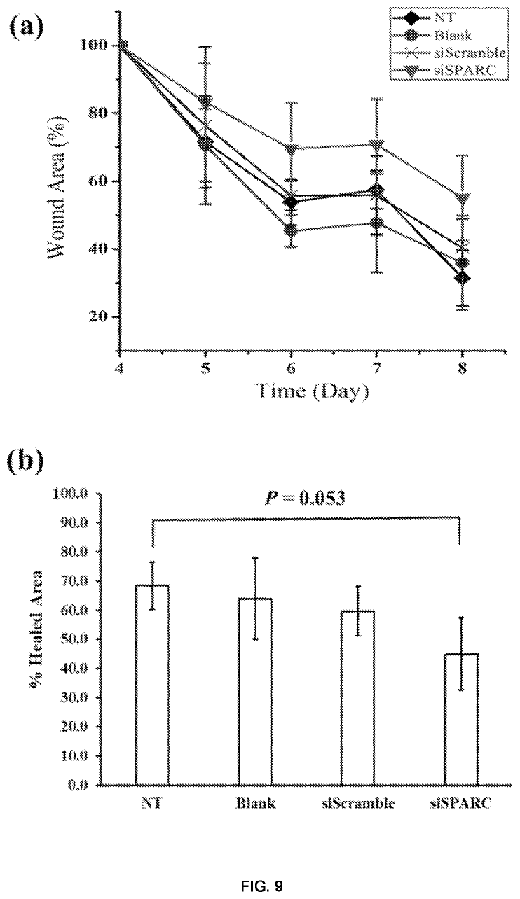

Physical observation (FIG. 8) showed that the wound treated with siSPARC treatment showed a relatively larger size at day 8 compared to non-treated wound and wound treated with blank or siScramble-loaded microneedles. The rate of wound area covered by full-thickness skin (FIG. 9a) is slightly slower for wound treated with siSPARC microneedle, where the healed area at day 8 (FIG. 9b) is slightly significant lower (45.0±12.5%, P=0.053) than non-treated wound (68.5±8.2%). Studies have shown that SPARC plays a role in wound repair by promoting fibroblast migration and granulation tissue formation (Basu, A. et al., BMC Cell Biol. 2001, 2, 1-9; and Wong, T. T. et al., Investig. Ophthalmol. Vis. Sci. 2009, 50, 3907). Inhibition of SPARC is associated with a reduction in MMP-2 activity, which is likely to help preserve wound functionality by delaying wound contraction and inhibition of fibrotic wound healing (Seet, L.-F. et al., PloS One 2010, 5, e9415). Thus, SPARC knockdown using siSPARC microneedles slowed down wound area covered by full-thickness skin, potentially decreasing the possibility of scar formation.

### Example 6. Wound Histological Examination Analysis

### Wound Histological Examination

Excised wounds were examined via H&E staining as previously described (Chong, H. C. et al., Mol. Ther. 2014, 22, 1593-1604) and PSR staining via PSR Stain Kit according to manufacturer's protocol (Abcam, U.S.). Stained wounds were then imaged via Zeiss Axioscan-Z1 (Carl Zeiss, Germany) at 20× magnification. Images were then analyzed for collagen quantification via ImageJ (plugin).

### Results and Discussion

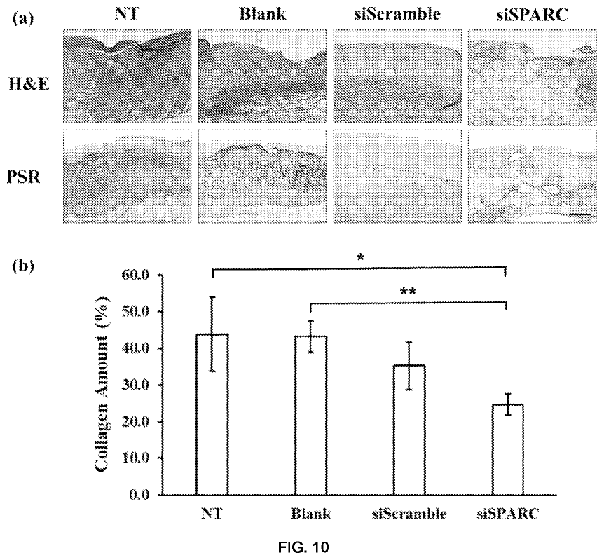

PSR staining (FIG. 10a) showed a reduction in collagen deposition at the wound site treated with siSPARC microneedles. When quantified using ImageJ (FIG. 10b), the wound treated with siSPARC microneedles showed significant lower amount of collagen (24.6±2.9%) as compared to non-treated wound (43.8±10.1%, P<0.05) and blank microneedles (43.3±4.4%, P<0.01). Reducing SPARC expression has been shown to reduce pro-fibrotic gene expressions, such as collagen I. SPARC knockdown has been shown to modulate fibroblast functions (Kim, B., Park, J. H. & Sailor, M. J., Adv. Mater. 2019, 31, 1903637) and prevent excessive collagen deposition, which is important for wound fibrosis. Therefore, siSPARC microneedles is a promising strategy in developing anti-scarring therapeutics by preventing excessive collagen deposition.

### Example 7. Wound Flow Cytometry Analysis

### Flow Cytometry

Excised wounds were processed with enzymatic digestion via Whole Skin Dissociation Kit according to the manufacturer's protocol (Miltenyi Biotec, U.S.) with Collagenase Ill (MP Biomedicals, U.S.) addition at 1 mg/mL during enzymatic digestion of tissues. Isolated single-cells were then stained for macrophages (APC-CD11b⁺ and FITC-F4/80⁺) and neutrophils (APC-CD11b⁺ and FITC-Ly6G⁺) to determine infiltration of immune cells and wound inflammation, before further analysis via flow cytometry using BD Accuri™ C6 Plus (BD Biosciences, U.S.). Post-FACS analysis was performed using FlowJo v10.0.7 (FlowJo LLC, U.S.).

### Results and Discussion

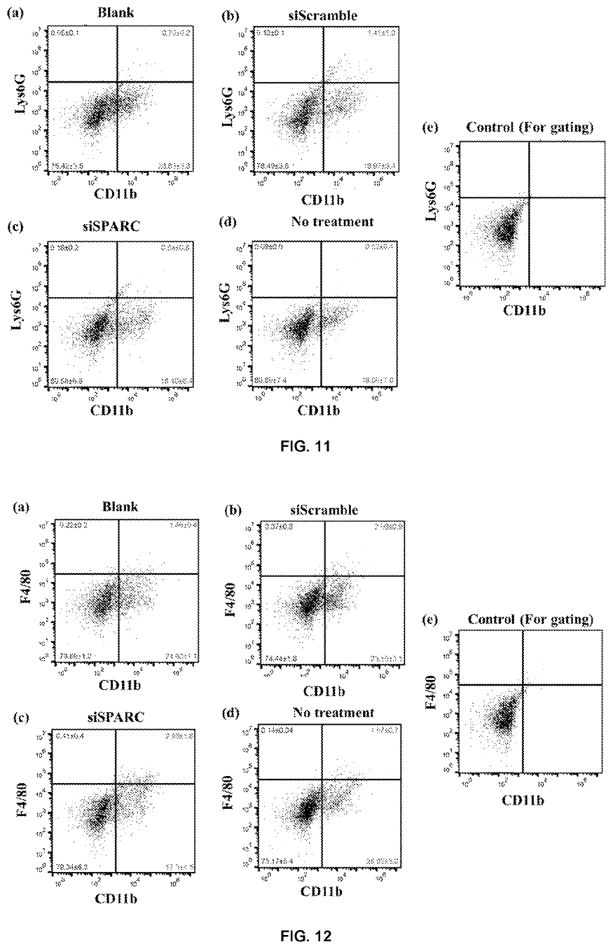

Immune response to the blank, siSPARC and siScramble microneedles were analyzed by flow cytometry for Lys6G, F4/80 and CD11b. CD11b is primarily expressed on granulocytes, monocytes/macrophages, dendritic cells, NK cells, and subsets of T and B cells. Neutrophils and macrophages are double positive for CD11b⁺Lys6G⁺ and CD11b⁺F4/80⁺, respectively. No significant difference was detected in the percentage of infiltrated CD11b⁺ myeloid-cells, neutrophils (FIG. 11) and macrophages (FIG. 12) between the different treatment groups when compared with no treatment control. Unstained cells were used for gating purpose. This observation suggests that the needles did not affect the in vivo immune response during wound healing.

### Example 8. In Vivo SPARC Gene Knockdown

RNA Extraction from Formalin-Fixed Paraffin-Embedded (FFPE) Tissues and GPCR

Samples embedded in paraffin were extracted and processed via FFPE RNA Purification Kit (Norgen Biotek, Canada) as per the manufacturer's protocol. Extracted RNAs were used for downstream qPCR as previously described (U, L. et al., mBio 2019, 10, e02469-18) using respective primers (Table 1) to validate SPARC silencing.

TABLE 1
Respective primers.

| Primer Name | Function | Sequence (5′-3′) |
|---|---|---|
| SPARC | Forward | CGAGACTTTGAGAAGAACTAC (SEQ |
| | | ID NO 13): |
| | Reverse | GGACAGGTACCCATCAATAG (SEQ |
| | | ID NO: 14) |
| TBP | Forward | GCTGGTTATCGGGAGTTGG (SEQ ID |
| | | NO: 15) |
| | Reverse | ACTGGCCTGGTGTCCTAGAG (SEQ |
| | | ID NO: 16) |

Results and discussion

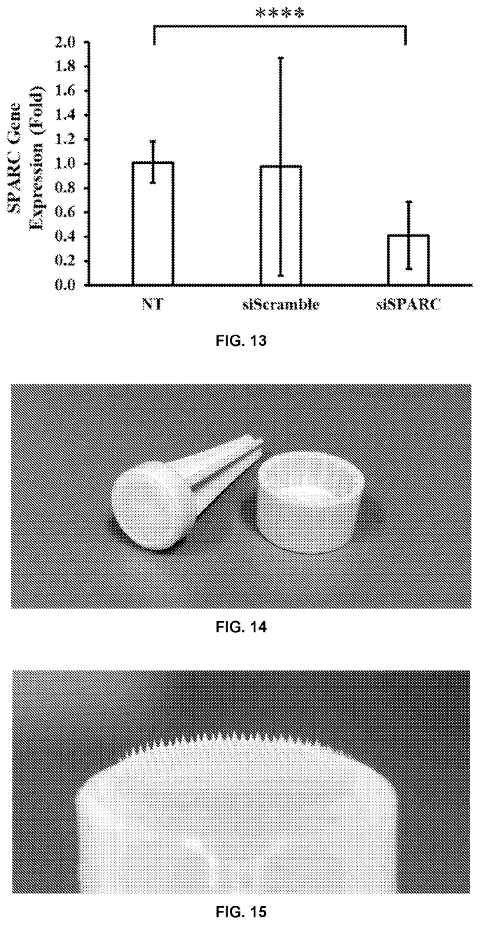

SPARC gene expression were quantified to confirm that the reduction in collagen deposition is due to the silencing of SPARC by the siSPARC microneedles. SPARC gene expression (FIG. 13) of wound tissue treated with siSPARC microneedles showed significant downregulation when compared with the non-treated wound (P<0.0001). The downregulation of SPARC gene expression by the siSPARC microneedles prevents collagen deposition and reduces scarring at the wound site.

### Example 9. Fabrication of Hard-Base and Flexible siRNA Microneedle Patches for Use in Humans

The microneedle patches have been fabricated in two forms: a hard-base patch and a flexible patch.

### Hard-Base Patch

For the hard-base patch (FIG. 14-15), microneedles composed of HA were attached to a plastic polymer base which has a handle for application to the skin. It came with a cap of the same hard plastic polymer material to prevent interaction of environmental moisture with HA, and to protect the microneedles from being blunted. The microneedles were 600 μm in length/height.

### Flexible Patch

For the flexible patch, the microneedles were localised to the central translucent portion of the patch, which is composed of HA. The borders are adhesive and the patch can be pasted onto the skin when the opaque strips are peeled off. The microneedle patch comprises a contiguous layer of siRNA and HA of 0.1-0.2 mm thick, which is fabricated into needles of 300 μm in length (FIG. 16-17). A closed-up photo showing the tips of flexible microneedle patch is depicted in FIG. 18.

### Example 10. Results on Use in Humans

Application of siRNA Microneedles in Humans

The two forms of siRNA microneedle patches (hard-based and flexible patches) were applied to healthy volunteers and an individual with healing wound for 3 h and 6 h. A dermoscope was used to image the microneedles in both unused and used patches of microneedles. High-definition optical coherence tomography (HD-OCT, Skintell, Agfa, Belgium) was used to image the skin to determine the presence and depth of penetration of the HA microneedles into the skin. A dermatologist subsequently evaluated the skin and wound both subjectively and objectively after application of microneedles, for evidence of side effects, such as irritation, allergy and infection.

### Results and Discussion

### Application Time for the Flexible Patch

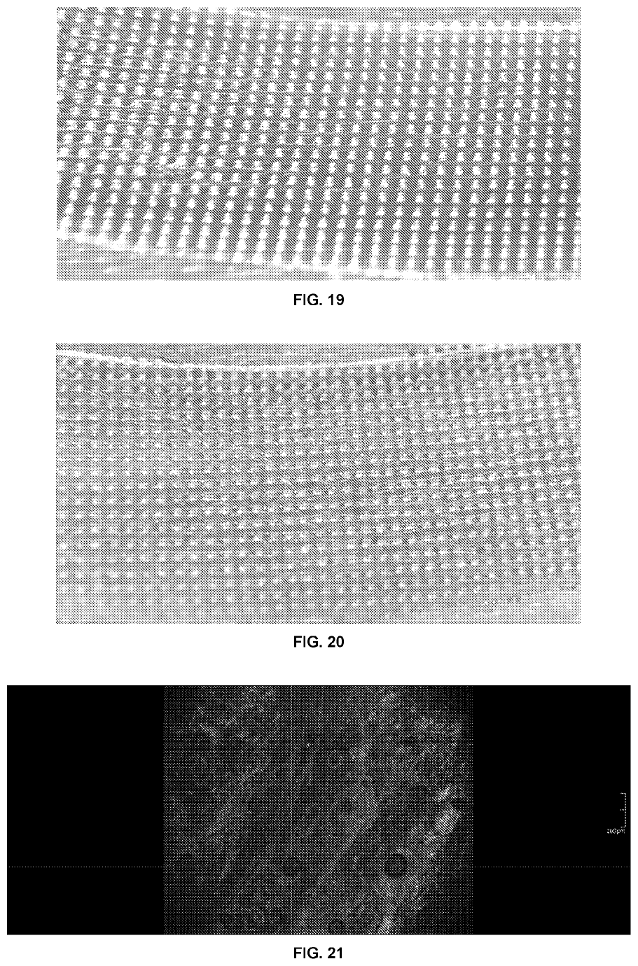

For the flexible patch, application on human skin for 3 h resulted in dissolution of about half of the microneedles (FIG. 19) while application for 6 h resulted in complete dissolution of the microneedles in most areas (FIG. 20). The rate of dissolution is dependent on the amount of moisture under the patch; if the person sweats during application, the microneedles dissolve at a much higher rate.

### Depth of Penetration

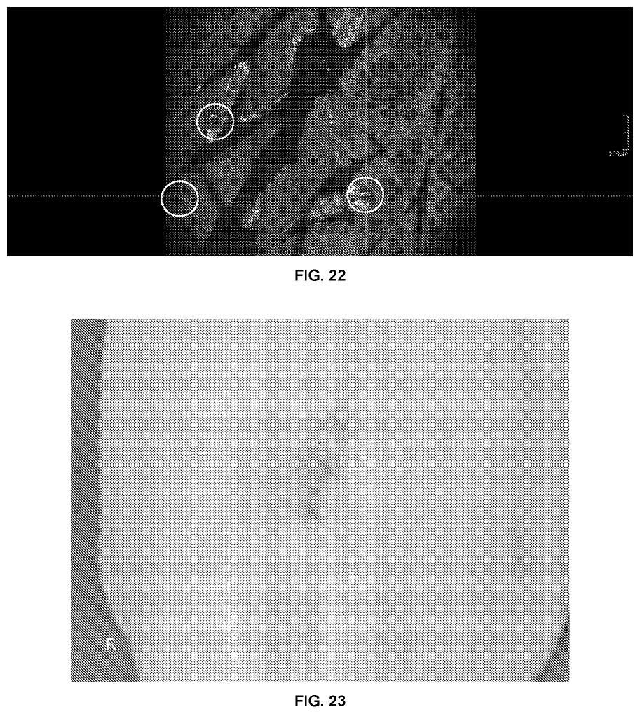

Imaging using HD-OCT was performed to determine skin penetration after application of the microneedles. Both the microneedle patches on the hard-based and flexible bases were visualized to have penetrated and deposited HA in the skin. For the hard-based patch, a mean penetration depth of 172.4±15.4 μm into the lower dermis can be visualized (FIG. 21). For the flexible patch, a mean penetration depth of 24.9±4.4 μm into the epidermis can be visualized (FIG. 22).

### Preliminary Safety Profile in Humans

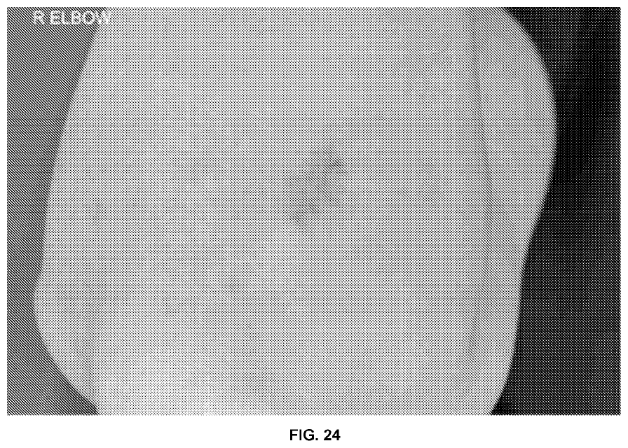

Apart from a prickly sensation during application, there were no side effects such as redness and itch to signify irritation and allergy, after application of both the flexible and hard-base patches on human subjects. The subject applied the hard-base patch from day 14 of the surgical wound (FIG. 23), once every 3 days. A month later, the wound was recovering well (FIG. 24) and the subject did not experience any side effects.

1. A dissolvable microneedle patch, comprising:
a substrate portion comprising a first biocompatible material; and
a plurality of microneedle structures extending from the substrate portion, where each microneedle structure comprises:
a base portion comprising a second biocompatible material; and
a tip portion, wherein the tip portion comprises:
a nanoplex formed from a positively charged biopolymer that incorporates a cell targeting moiety and an siRNA; and
a third biocompatible material.

2. The dissolvable microneedle patch according to claim 1, wherein the first biocompatible material is selected from one or more of the group consisting of hyaluronic acid, or other dissolvable and biocompatible polymers, cellulose derivatives and synthetic polymers.

3. The dissolvable microneedle patch according to claim 1, wherein the second biocompatible material is selected from one or more of the group consisting of hyaluronic acid, or other dissolvable and biocompatible polymers.

4. The dissolvable microneedle patch according to claim 1, wherein the first and the second biocompatible materials are the same biocompatible material.

5. The dissolvable microneedle patch according to claim 1, wherein the third biocompatible material is selected from one or more of the group consisting of hyaluronic acid, or other dissolvable and biocompatible polymers.

6. The dissolvable microneedle patch according to claim 1, wherein the first, second and third biocompatible materials are the same biocompatible material.

7. The dissolvable microneedle patch according to claim 1, wherein the positively charged biopolymer is formed from:
a heterobifunctional polymer comprising a first set of functional groups that are positively charged and a second set of functional groups that are negatively charged in an aqueous environment; and
a molecule conjugated to the heterobifunctional polymer, where the molecule is conjugated by a functional group that forms a bond with the second set of functional groups of the heterobifunctional polymer, so as to reduce the number of the second functional groups in the second set of functional groups, thereby providing the positively charged biopolymer.

8. The dissolvable microneedle patch according to claim 7, wherein the heterobifunctional polymer is selected from one or more of gelatin, collagen, silk fibroin, elastin and H₂N-PEG-CO₂H.

9. The dissolvable microneedle patch according to claim 8, wherein the heterobifunctional polymer is gelatin.

10. The dissolvable microneedle patch according to claim 7, wherein the functional group in the molecule conjugated to the heterobifunctional polymer that forms a bond with the second set of functional groups of the heterobifunctional polymer is selected from one or more of amide, amino, and ammonium.

11. The dissolvable microneedle patch according to claim 7,
wherein the molecule conjugated to the heterobifunctional polymer is selected from one or more of 4-hydroxybenzylamine, dopamine, and tyramine.

12. The dissolvable microneedle patch according to claim 7, wherein the cell targeting moiety is selected from one or more of gelatin, transferrin, cyclic-Arginine-Glycine-Aspartic acid (RGD) tripeptide, skin protein-derived peptides, and folic acid.

13. The dissolvable microneedle patch according to claim 1, wherein the siRNA is selected from one or more of the group consisting of:
```
(a) siTGFβ1-337:
  (SEQ ID NO: 1)
5′-GGUGGAAACCCACAACGAATT-3′;
(b) SiFAK:
  (SEQ ID NO: 2)
5′-GCGAAATCCA-TAGCAGGCCACTATAGTGAGTCGTATTACC-3′;
(c) siSphk2:
  (SEQ ID NO: 3)
5′-CAGGATTGCGCTCGCTTTCAT-3′;
(d) siRunx2:
  (SEQ ID NO: 4)
5′-CAGAAGAATGGTACAAATCCAAG-3′;
(e) sieIF3a:
  (SEQ ID NO: 5)
5′-GCAGATGGTCTTAGATATA-3′;
(f) SiEDB:
  (SEQ ID NO: 6)
5′-AAGGTATCCCTATTTTTGAAGCCTGTCTC-3′;
(g) siRe1A:
  (SEQ ID NO: 7)
5′-GGU GCA GAA AGA CAU UdTdT-3′;
(h) siIL-10:
  (SEQ ID NO: 8)
5′-UUUAGCUACUACCGCAGCGdTdT-3′;
(i) siGPNMB:
  (SEQ ID NO: 9)
5′-GCACGGGUUUCUAUAAACAdTdT-3′;
(j) SISPARC:
  (SEQ ID NO: 10)
5′-AACAAGACCUUCGACUCUUCC-3′.
```

14. The dissolvable microneedle patch according to claim 13, wherein the siRNA is siSPARC: 5′-AACAAGACCUUCGACUCUUCC-3′ (SEQ ID NO:10).

15. The dissolvable microneedle patch according to claim 1, wherein the plurality of microneedle structures extending from the substrate portion have a height of from 100 μm to 2.5 mm.

16. (canceled)

17. (canceled)

18. (canceled)

19. A method of treatment of a skin disorder or condition, which method comprises the administration of a dissolvable microneedle patch according to claim 1.

20. The method according to claim 19, wherein the skin condition or disorder is selected from one or more of the group consisting of scarring, keloid formation, eczema, and hyperpigmentation.

21. The uses and the method according to claim 20, wherein the skin condition or disorder is hypertrophic scarring.

22. The uses and the method according to claim 21, wherein the dissolvable microneedle patch is applied to the skin after wound union has occurred.

23. The dissolvable microneedle patch according to claim 1, wherein the third biocompatible material is hyaluronic acid.


SEQUENCE LISTING (16 sequences)

SEQ ID NO: 1 - RNA, 21 nt, Artificial Sequence
  siRNA
  misc_feature (20)..(21): n is thymine
gguggaaacccacaacgaann

SEQ ID NO: 2 - RNA, 40 nt, Artificial Sequence
  siRNA
  misc_feature (7)..(7): n is thymine
  misc_feature (11)..(11): n is thymine
  misc_feature (22)..(22): n is thymine
  misc_feature (24)..(24): n is thymine
  misc_feature (27)..(27): n is thymine
  misc_feature (31)..(31): n is thymine
  misc_feature (34)..(34): n is thymine
  misc_feature (36)..(37): n is thymine
gcgaaanccanagcaggccacnanagngagncgnannacc

SEQ ID NO: 3 - RNA, 21 nt, Artificial Sequence
  siRNA
  misc_feature (6)..(7): n is thymine
  misc_feature (12)..(12): n is thymine
  misc_feature (16)..(18): n is thymine
  misc_feature (21)..(21): n is thymine
cagganngcgcncgcnnncan

SEQ ID NO: 4 - RNA, 23 nt, Artificial Sequence
  siRNA
  misc_feature (9)..(9): n is thymine
  misc_feature (12)..(12): n is thymine
  misc_feature (18)..(18): n is thymine
cagaagaanggnacaaanccaag

SEQ ID NO: 5 - RNA, 19 nt, Artificial Sequence
  siRNA
  misc_feature (6)..(6): n is thymine
  misc_feature (9)..(9): n is thymine
  misc_feature (11)..(12): n is thymine
  misc_feature (16)..(16): n is thymine
  misc_feature (18)..(18): n is thymine
gcaganggncnnaganana

SEQ ID NO: 6 - RNA, 29 nt, Artificial Sequence
  siRNA
  misc_feature (5)..(5): n is thymine
  misc_feature (7)..(7): n is thymine
  misc_feature (11)..(11): n is thymine
  misc_feature (13)..(17): n is thymine
  misc_feature (24)..(24): n is thymine
  misc_feature (26)..(26): n is thymine
  misc_feature (28)..(28): n is thymine
aaggnancccnannnnngaagccngncnc

SEQ ID NO: 7 - RNA, 18 nt, Artificial Sequence
  siRNA
  misc_feature (17)..(18): n is thymidine
ggugcagaaagacauunn

SEQ ID NO: 8 - RNA, 21 nt, Artificial Sequence
  siRNA
  misc_feature (20)..(21): n is thymidine
uuuagcuacuaccgcagcgnn

SEQ ID NO: 9 - RNA, 21 nt, Artificial Sequence
  siRNA
  misc_feature (20)..(21): n is thymidine
gcacggguuucuauaaacann

SEQ ID NO: 10 - RNA, 21 nt, Artificial Sequence
  siRNA
aacaagaccuucgacucuucc

SEQ ID NO: 11 - protein, 5 aa, Homo sapiens
KTTKS

SEQ ID NO: 12 - RNA, 21 nt, Artificial Sequence
  siRNA
gcucacagcucaauccuaauc

SEQ ID NO: 13 - DNA, 21 bp, Artificial Sequence
  primer
cgagactttgagaagaactac

SEQ ID NO: 14 - DNA, 20 bp, Artificial Sequence
  reverse primer
ggacaggtacccatcaatag

SEQ ID NO: 15 - DNA, 19 bp, Artificial Sequence
  forward primer
gctggttatcgggagttgg

SEQ ID NO: 16 - DNA, 20 bp, Artificial Sequence
  reverse primer
actggcctggtgtcctagag